(19) United States
(12) Patent Application Publication
(10) Pub. No.: US 2018/0088123 A1
(43) Pub. Date: Mar. 29, 2018

(54) ORGANIC ELECTROLUMINESCENT MATERIALS AND ORGANIC ELECTROLUMINESCENT DEVICES

(71) Applicant: Shenzhen China Star Optoelectronics Technology Co., Ltd., Shenzhen, Guangdong (CN)

(72) Inventor: Huafei XIE, Shenzhen, Guangdong (CN)

(73) Assignee: Shenzhen China Star Optoelectronics Technology Co., Ltd., Shenzhen, Guangdong (CN)

(21) Appl. No.: 15/129,564

(22) PCT Filed: Jul. 5, 2016
(86) PCT No.: PCT/CN2016/088498

§ 371 Date: Sep. 27, 2016

(30) Foreign Application Priority Data
May 20, 2016 (CN) 201610340847.3

(51) Int. Cl. G01N 33/58; G01N 33/52; A61K 49/00; C07F 9/54; C07D 209/08; C07D 217/04; C09K 11/07

(52) U.S. Cl. CPC G01N 33/582; G01N 33/52; A61K 49/0023; C09K 11/07; C07D 209/08; C07D 217/04; C07F 9/5449

(57) ABSTRACT

The present application discloses an organic electroluminescent material, a molecular formula thereof is Da-π-Ac, wherein Da is an aggregation-induced emission group, π is a conjugated bond, and Ac is a strong electron-withdrawing group. The organic electroluminescent material of the present application has a simple preparation method, high yield, good thermodynamic stability, can cover the visible region, have excellent electroluminescence effect, and can prepare undoped type of organic electroluminescent devices.

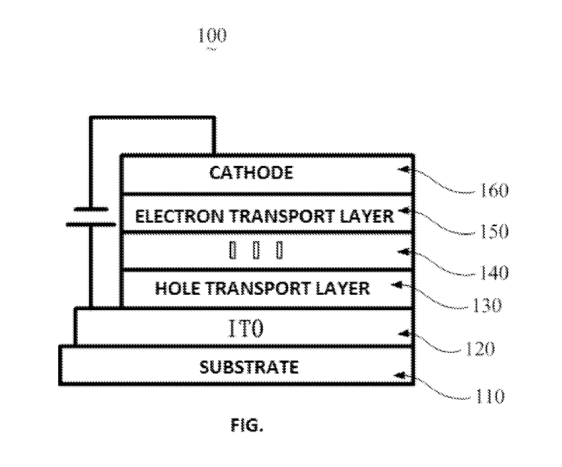

### FIELD OF THE INVENTION

The present application relates to a display technical field; especially to an organic electroluminescent material and a related organic electroluminescent device.

### BACKGROUND OF THE INVENTION

With the development of scientific technology, people have made increasing demands on the display devices of contrast, color change, resolution, cost, energy consumption and quality. Due to various high energy consumption, high cost and other shortcomings, liquid crystal display devices have become increasingly difficult to meet the demands of people, while the organic electroluminescent technology obtains a great development, an organic Light-Emitting Diode (OLED) has advantages of self-luminescence, low-voltage DC drive, wide viewing angle, fast response, wide operating temperature range and flexible display, etc.; therefore, it has become a trend of scientific technology development that the organic electroluminescent display replaces the liquid crystal display device.

In the organic electroluminescent technology, a choice of an organic electroluminescent material is crucial, and a property thereof is one of the key factors that determine the device performance. Currently, the organic electroluminescent material substantially utilizes compounds or polymers of organic small molecules, which has chemical modification, wide option range, easy purification, high fluorescence quantum efficiency, and it can produce wide color specificity of red, green, blue and various colors.

Most of organic dyes in the solid-state exist a problem of concentration quench, that would cause the devices in drawbacks of emission peak broadening, spectra redshift, fluorescence quantum drop; therefore, they are generally at lowest concentration to be doped in a host having certain carrier property, trace of the organic fluorescence dye are dispersed in the matrix of host light emitter by using the principle of energy transfer, so that guest molecules can emit light through the transfer of excitation energy. Therefore, the currently reported high performance organic electroluminescent devices mostly use the preparation method of doping light emitting layer.

However, such the preparation method of doping requires precise regulation to ensure the accuracy of doping concentration that would increase the manufacture cost and be unfavorable for industrialization of the organic electroluminescent technology. Simultaneously, the phase separation and energy transfer of host-guest in the doped device also affect the performance and stability of the device.

Therefore, in comparison with the doped type of organic electroluminescent device, the undoped type of organic electroluminescent device can avoid the effect of doping concentration, that strengthens the stability and repeatability of the device, effectively extends the life of the device, and reduces the difficulty of processing technique, is thus suitable for large-scale commercial production. However, in the undoped type of organic electroluminescent device, a high performance undoped light emitting material needs to have critical characteristics such as high efficiency solid-state light-emitting, good light color and stronger carrier transport capability, and the high performance undoped light emitting material is very scarce; therefore, it is urgent to invent an organic solid-state undoped electroluminescent material and an organic electroluminescent device corresponding thereto.

### SUMMARY OF THE INVENTION

A main technical problem the present application desires to solve is to provide an organic electroluminescent material and an organic electroluminescent device corresponding thereto, which has a simple preparation method, high yield, good thermodynamic stability, can cover the visible region, have excellent electroluminescence effect, and can prepare an undoped type of organic electroluminescent device.

To solve the aforesaid technical problem, a solution utilized by the present application is to provide an organic electroluminescent material, a molecular formula thereof is Da-π-Ac, wherein Da is an aggregation-induced emission group, π is a conjugated bond, and Ac is a strong electron-withdrawing group;

Da has a structural formula as follow:

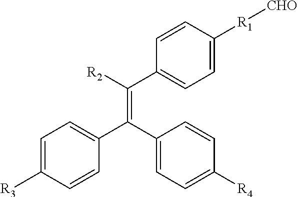

R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl;

Ac is selected from one of following structures:

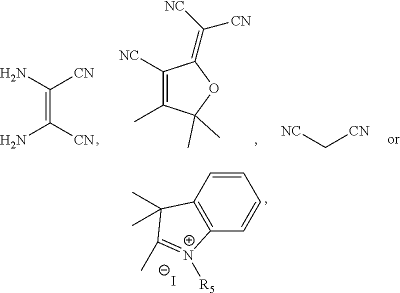

wherein R5 is selected from C1˜5 alkyl or nitro(-NO₂).

Wherein Da is selected from one of following structures:

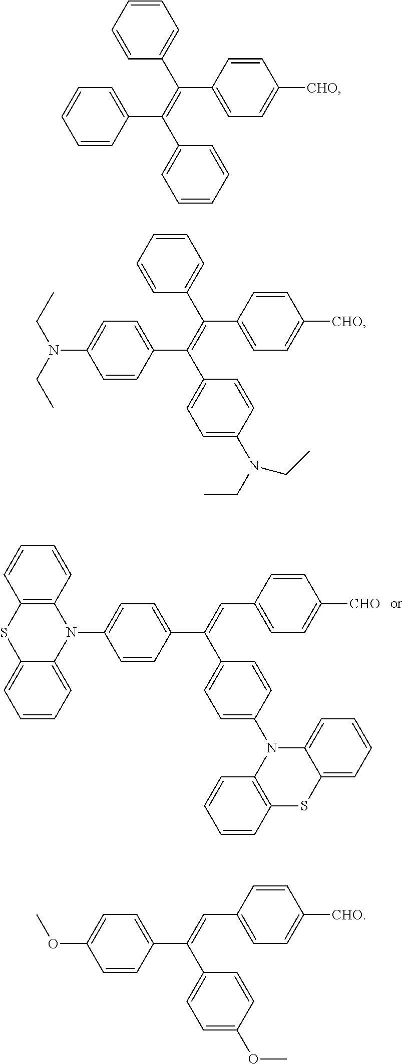

Wherein the organic electroluminescent material is manufactured by reacting the aggregation-induced emission group having aromatic aldehyde and the strong electron-withdrawing group having active methyl in a weak base environment,

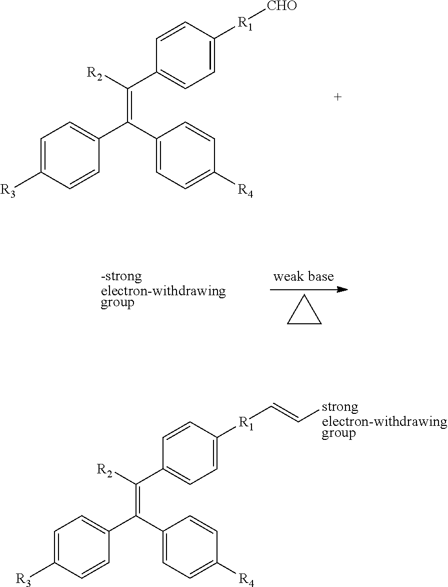

wherein R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl.

Wherein the organic electroluminescent material is manufactured by reacting the aggregation-induced emission group having aromatic aldehyde and the strong electron-withdrawing group having amino in a weak acid environment,

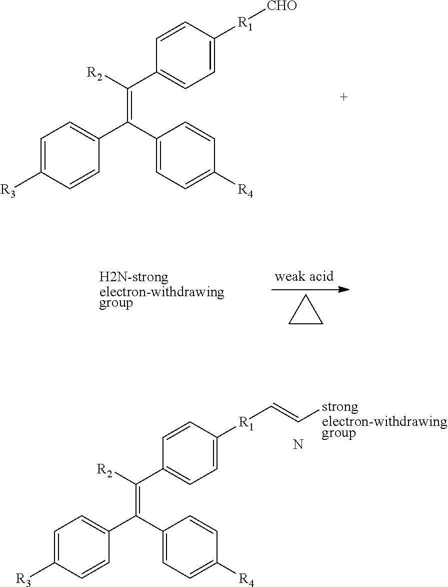

wherein R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl.

To solve the aforesaid technical problem, another solution utilized by the present application is to provide an organic electroluminescent material, a molecular formula of the organic electroluminescent material is Da-π-Ac, wherein Da is an aggregation-induced emission group, π is a conjugated bond, and Ac is a strong electron-withdrawing group.

Wherein Da has a structural formula as follow:

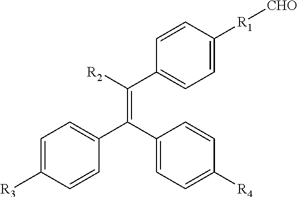

R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl.

Wherein Da is selected from one of following structures:

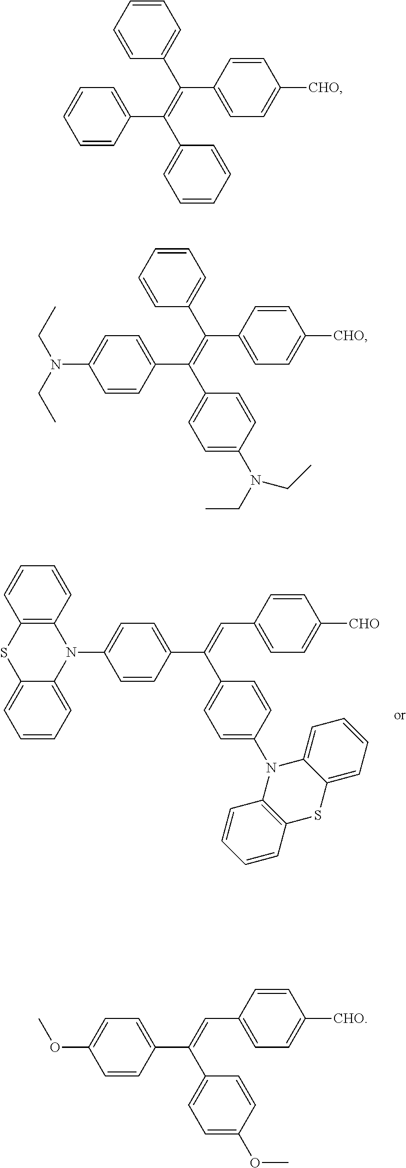

Wherein Ac is selected from one of following structures:

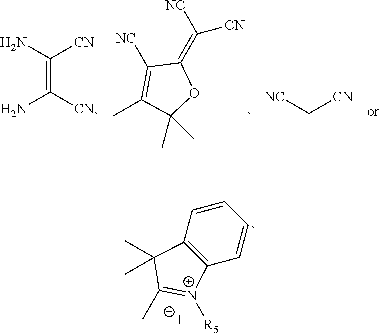

wherein R5 is selected from C1˜5 alkyl or nitro(-NO₂).

Wherein the organic electroluminescent material is manufactured by reacting the aggregation-induced emission group having aromatic aldehyde and the strong electron-withdrawing group having active methyl in a weak base environment,

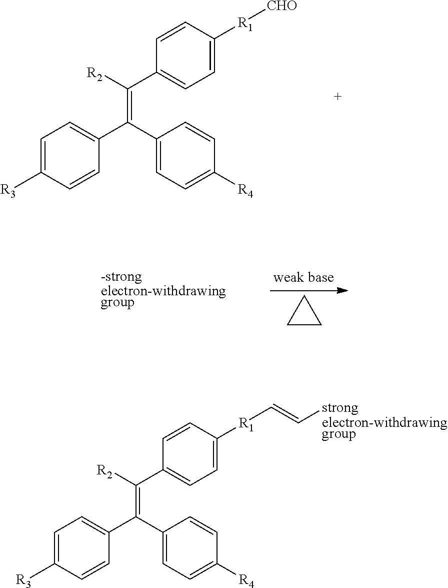

wherein the organic electroluminescent material is manufactured by reacting the aggregation-induced emission group having aromatic aldehyde and the strong electron-withdrawing group having amino in a weak acid environment,

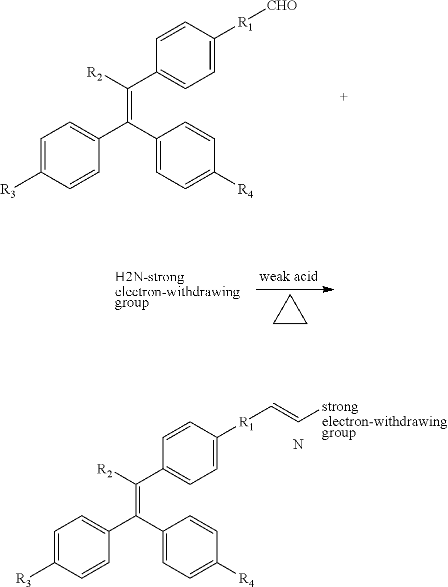

wherein a molecular formula of the organic electroluminescent material is selected from one of following structures:

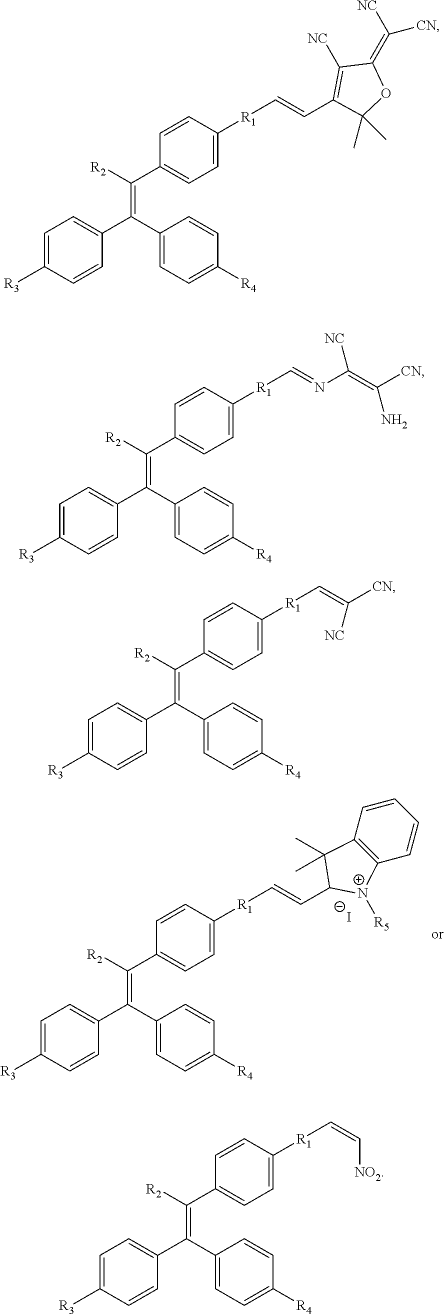

To solve the aforesaid problem, another solution utilized by the present application is to provide an organic electroluminescent device, which includes: a substrate; and an anode, a hole transport layer, a light-emitting layer, an electron transport layer and a cathode sequentially disposed on the substrate; wherein a molecular formula of the light-emitting layer is Da-π-Ac, Da is an aggregation-induced emission group, π is a conjugated bond, and Ac is a strong electron-withdrawing group.

Advantages of the present application are, different from the situation in the prior art, that a method of preparing the organic electroluminescent material of the present application is simple, and all the raw materials can be obtained in high yield. Additionally, due that the skeleton of the electroluminescent material of the present application has benzene rings, which can present higher thermodynamic stability. A fluorescent material covering the visible region can be effectively manufactured by the molecular structure of Da-π-Ac, such molecular structure can produce very strong fluorescence in the solid-state and has good electroluminescent effect, therefore, the undoped type of organic electroluminescent device can be prepared by using such material, so as to avoid the effect of doping concentration, that strengthens the stability and repeatability of the device, effectively extends the life of the device, and reduces the difficulty of processing technique, is thus suitable for large-scale commercial production.

### BRIEF DESCRIPTION OF THE DRAWINGS

FIGURE schematically illustrates an organic electroluminescent device of an embodiment of the present application.

### DETAILED DESCRIPTION OF PREFERRED EMBODIMENTS

The present application will be described in detail by referring the following embodiments with the accompanying drawing.

The present application provides an organic solid-state undoped electroluminescent material, a molecular thereof is Da-π-Ac, wherein Da is an aggregation-induced emission group, π is a conjugated bond, and Ac is a strong electron-withdrawing group.

Wherein Da can utilizes a structural formula as follow:

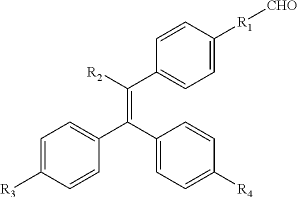

R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl.

In an embodiment of the present application, Da can be selected from any one of following structures:

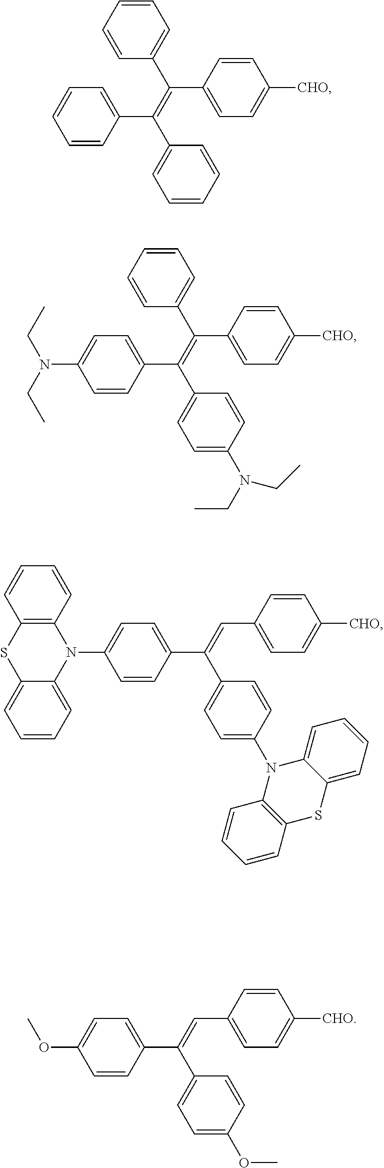

Ac can be selected from any one of following structures:

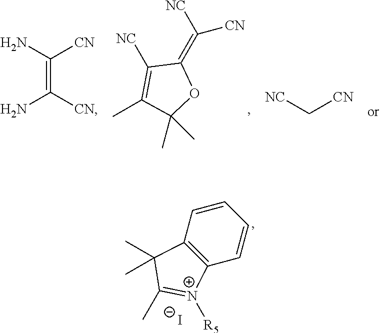

wherein R5 is selected from C1˜5 alkyl or nitro(-NO₂).

In an embodiment of the present application, the molecular formula of the organic electroluminescent material can be selected from any one of following structures:

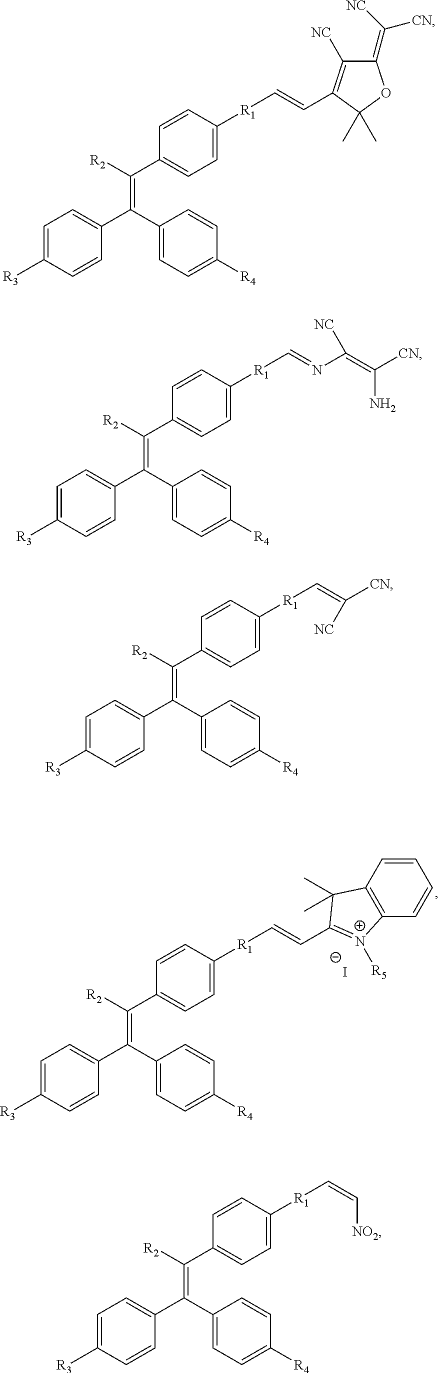

wherein R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl; and R5 is selected from C1˜5 alkyl or nitro(-NO₂).

The organic electroluminescent material of the present application can be manufactured by reacting the aggregation-induced emission group having aromatic aldehyde and the strong electron-withdrawing group having active methyl in a weak base environment, a reaction formula thereof is as follow:

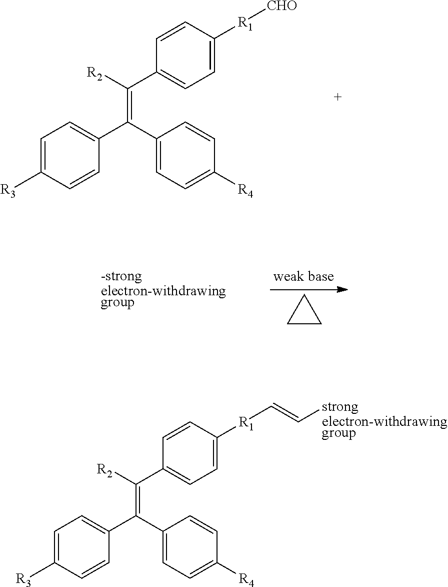

alternatively, the organic electroluminescent material of the present application can be manufactured by reacting the aggregation-induced emission group having aromatic aldehyde and the strong electron-withdrawing group having amino in a weak acid environment, a reaction formula thereof is as follow:

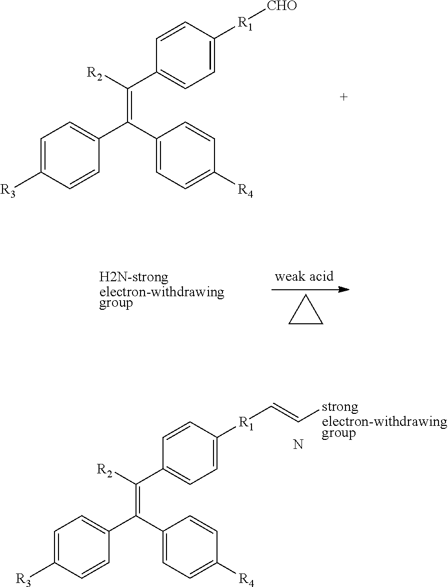

In an embodiment of the present application, Da can select tetraphenylethylene or triphenylethylene derivatives containing aromatic aldehyde, so as to allow the aromatic aldehyde react with the active methyl or amino; thereby, a compound having ALE-π-strong electron-withdrawing group structure of good thermal stability and solid-state quantum yield is prepared.

In the compound of such structure, due to conjugate introduction of the strong electron-withdrawing group, which electron affinity is significantly increased, intra-molecular charge transfer is enhanced, band gap of the molecules is adjusted, light absorption/emission range of the molecule are expanded, etc., so as to become an excellent performance electroluminescent material that holes and electrons are able to efficiently inject and transport.

The present application further provides an organic electroluminescent device prepared by utilizing the aforesaid organic electroluminescent material. FIGURE schematically illustrates an organic electroluminescent device of a embodiment of the present application.

As shown in FIGURE, the organic electroluminescent device 100 of the present application includes a substrate 110, an anode 120, a hole transport layer 130, a light-emitting layer 140, an electron transport layer 150 and a cathode 160. Wherein the anode 120, the hole transport layer 130, the light-emitting layer 140, the electron transport layer 150 and the cathode 160 are sequentially disposed on the substrate 110.

Wherein the anode can be made by using a material of indium tin oxide (ITO); the hole transport layer 130 can be made by using a material of NPB(N,N′-di(1-naphthyl)-N,N′-diphenyl-1,1′-biphenyl-4-4′-diamine); the light emitting layer 140 can be made by using the aforesaid organic electroluminescent material having Da-π-Ac structure; the electron transport layer 150 can be made by using a material of Alq3(tris(8-quinolinol)aluminum); the cathode 160 can be made by using a material of lithium fluoride (LiF) or aluminum (Al).

The method of preparing the organic electroluminescent material of the present application is simple, and all the raw materials can be obtained in high yield. Additionally, due that the skeleton of the electroluminescent material of the present application has benzene rings, which can present higher thermodynamic stability. A fluorescent material covering the visible region can be effectively manufactured by the molecular structure of Da-π-Ac, such molecular structure can produce very strong fluorescence in the solid-state and has good electroluminescent effect, therefore, the undoped type of organic electroluminescent device can be prepared by using such material, so as to avoid the effect of doping concentration, that strengthens the stability and repeatability of the device, effectively extends the life of the device, and reduces the difficulty of processing technique, is thus suitable for large-scale commercial production.

The above description is only for the embodiment of the present application, but not limit the patent scope of the present application, any modifications and arrangements by utilizing the technical programs and ideas of the present application, directly or indirectly applied to relative technical fields, the scope of the appended claims of the present application should encompass all such modifications and arrangements.

What is claimed is:

1. An organic electroluminescent material, in which a molecular formula of the organic electroluminescent material is Da-π-Ac, wherein Da is an aggregation-induced emission group, π is a conjugated bond, and Ac is a strong electron-withdrawing group;
Da has a structural formula as follow: 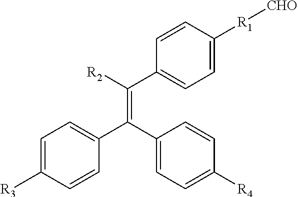
wherein R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl;
Ac is selected from one of following structures: 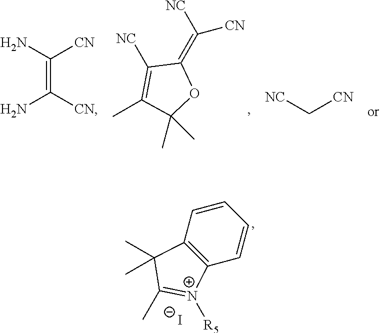
wherein R5 is selected from C1˜5 alkyl or nitro(-NO₂).

2. The organic electroluminescent material according to claim 1, wherein Da is selected from one of following structures:

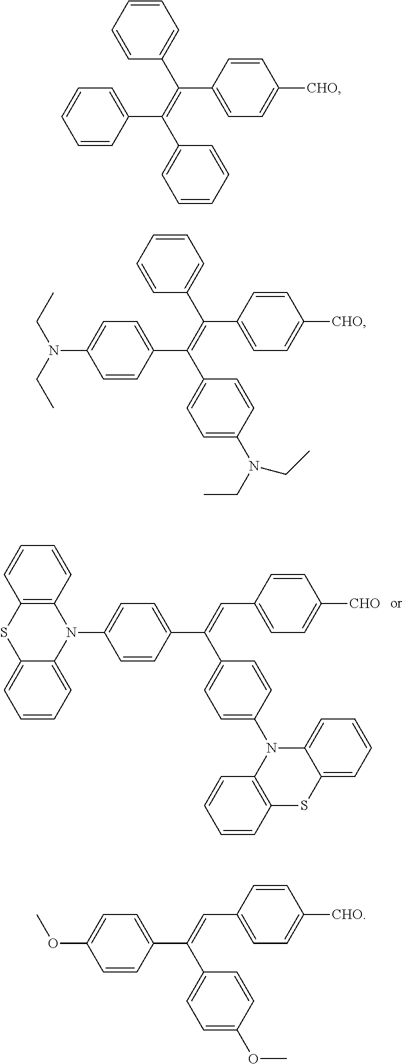

3. The organic electroluminescent material according to claim 1, wherein the organic electroluminescent material is manufactured by reacting the aggregation-induced emission group having aromatic aldehyde and the strong electron-withdrawing group having active methyl in a weak base environment, 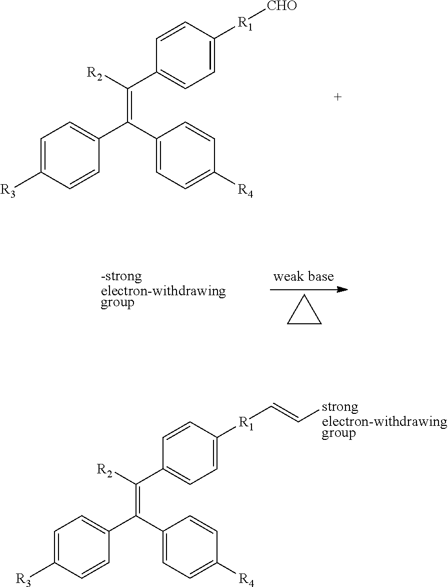
wherein R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl.

4. The organic electroluminescent material according to claim 1, wherein the organic electroluminescent material is manufactured by reacting the aggregation-induced emission group having aromatic aldehyde and the strong electron-withdrawing group having amino in a weak acid environment, 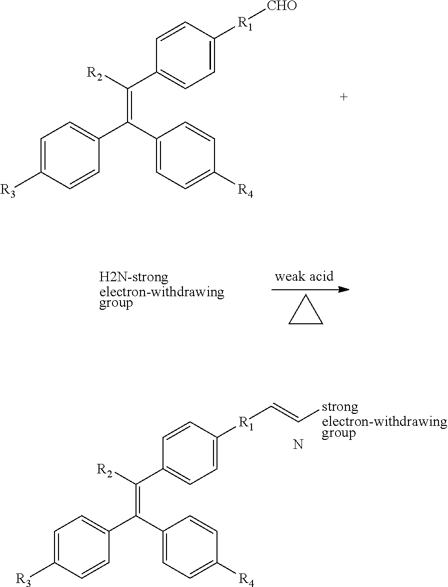
wherein R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl.

5. An organic electroluminescent material, in which a molecular formula of the organic electroluminescent material is Da-π-Ac, wherein Da is an aggregation-induced emission group, π is a conjugated bond, and Ac is a strong electron-withdrawing group.

6. The organic electroluminescent material according to claim 5, wherein Da has a structural formula as follow: 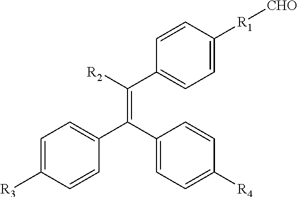
wherein R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl.

7. The organic electroluminescent material according to claim 6, wherein Da is selected from one of following structures:

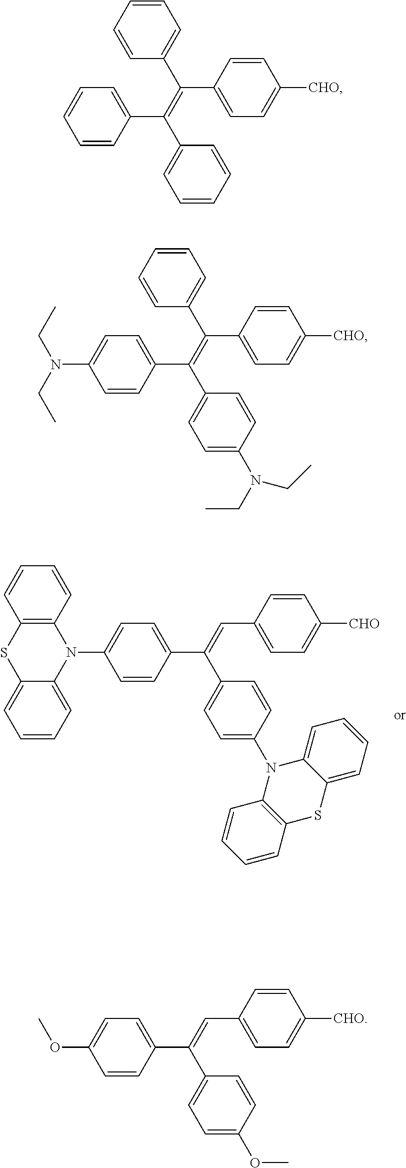

8. The organic electroluminescent material according to claim 5, wherein Ac is selected from one of following structures: 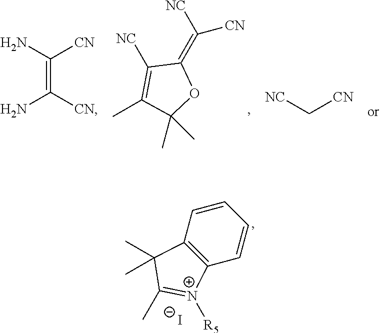
wherein R5 is selected from C1˜5 alkyl or nitro(-NO₂).

9. The organic electroluminescent material according to claim 5, wherein the organic electroluminescent material is manufactured by reacting the aggregation-induced emission group having aromatic aldehyde and the strong electron-withdrawing group having active methyl in a weak base environment, 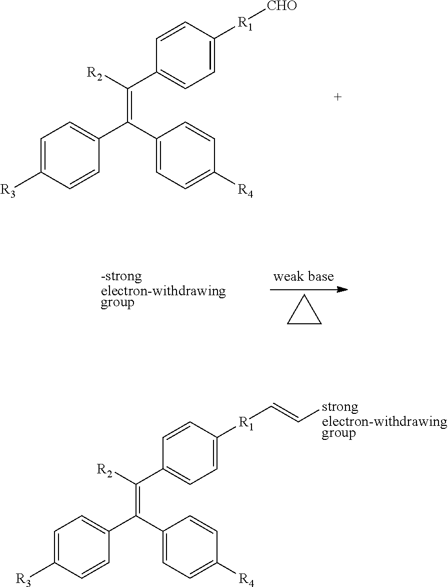
wherein R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl.

10. The organic electroluminescent material according to claim 5, wherein the organic electroluminescent material is manufactured by reacting the aggregation-induced emission group having aromatic aldehyde and the strong electron-withdrawing group having amino in a weak acid environment, 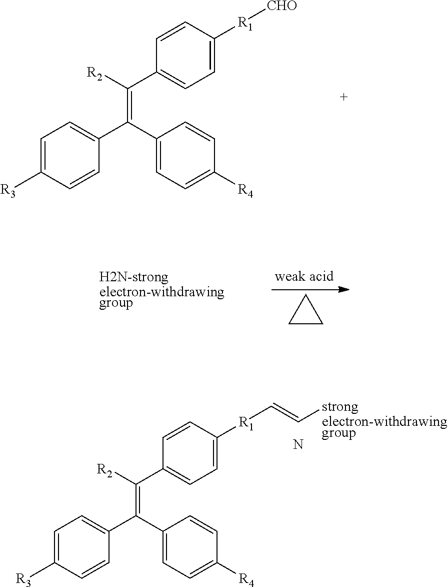
wherein R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl.

11. The organic electroluminescent material according to claim 5, wherein a molecular formula of the organic electroluminescent material is selected from one of following structures: 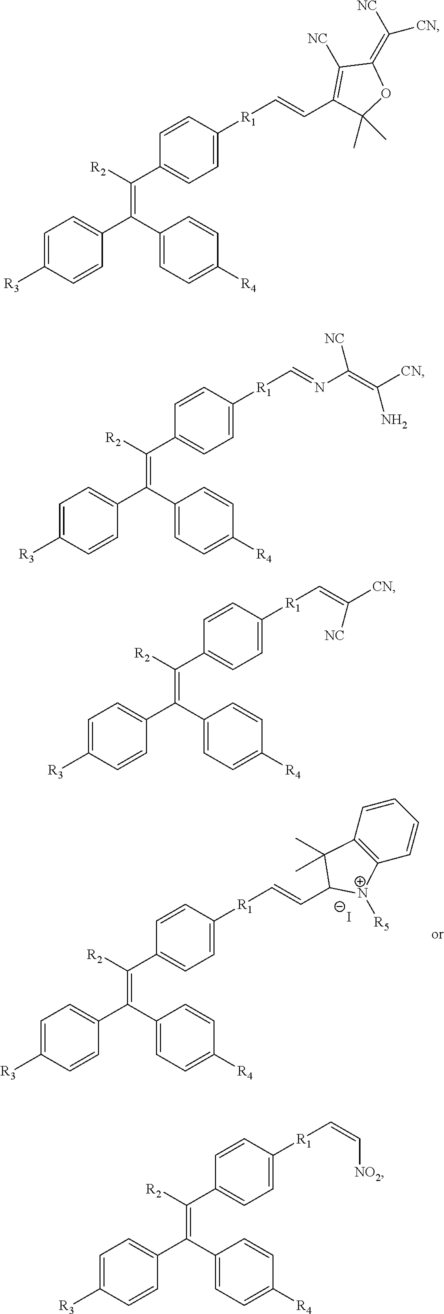
wherein R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl; and R5 is selected from C1˜5 alkyl or nitro(-NO₂).

12. An organic electroluminescent device, which comprises: a substrate; and an anode, a hole transport layer, a light-emitting layer, an electron transport layer and a cathode sequentially disposed on the substrate; wherein a molecular formula of the light-emitting layer is Da-π-Ac, Da is an aggregation-induced emission group, π is a conjugated bond, and Ac is a strong electron-withdrawing group.

13. The organic electroluminescent device according to claim 12, wherein Da has a structural formula as follow: 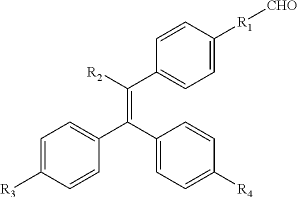
wherein R1 is a direct single bond or an aromatic ring; R2 is a hydrogen atom or a benzene ring; and R3 and R4 are respectively selected from at least one of hydrogen atom, C1˜8 alkyl, C1˜4 alkoxy, aryl and carbazolyl.

14. The organic electroluminescent device according to claim 12, wherein Ac is selected from one of following structures: 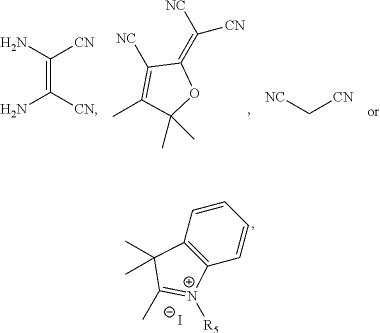
wherein R5 is selected from C1˜5 alkyl or nitro(-NO₂).
